(19) United States
(12) Patent Application Publication
(10) Pub. No.: US 2024/0210400 A1
(43) Pub. Date: Jun. 27, 2024

(54) METHODS FOR DETECTING OR TREATING ENDOMETRIAL AND OVARIAN HYPERPROLIFERATIVE DISORDERS

(71) Applicant: Temple Therapeutics BV, Geleen (NL)

(72) Inventor: GHASSAN SAED, Temple, TX (US)

(21) Appl. No.: 18/555,933

(22) PCT Filed: Apr. 19, 2022
(86) PCT No.: PCT/IB2022/053647

§ 371 Date: Oct. 18, 2023

Related U.S. Application Data
(60) Provisional application No. 63/176,852, filed on Apr. 19, 2021.
(60) Provisional application No. 63/277,438, filed on Nov. 9, 2021.

(51) Int. Cl. G01N 33/573; G01N 33/574; G01N 33/84

(52) U.S. Cl. CPC G01N 33/573; G01N 33/57449; G01N 33/84; G01N 2333/908

(57) ABSTRACT

The current disclosure provides sensitive diagnostic methods that do not require invasive laparoscopic surgery. It was found that monomeric myeloperoxidase (MPO) was found in cells, tissues, and the sera of patients with endometriosis. This novel biomarker can be used to detect ovarian and endometrial hyperproliferative disorders. Accordingly, aspects of the disclosure relate to a method for evaluating a subject comprising detecting monomeric myeloperoxidase (MPO) in a biological sample from the subject. Also disclosed is a method for treating a subject with an endometrial or ovarian hyperproliferative disorder, the method comprising administering an treatment for the endometrial or ovarian hyperproliferative disorder to a subject that has had the level of monomeric MPO evaluated in a biological sample from the subject.

### CROSS-REFERENCE TO RELATED APPLICATIONS

This application claims the benefit of priority of U.S. Provisional Patent Application No. 63/176,852 filed Apr. 19, 2021, and U.S. Provisional Patent Application No. 63/277,438 filed Nov. 9, 2021, which are hereby incorporated by reference in their entirety.

### BACKGROUND OF THE INVENTION

### I. Field of the Invention

The present invention relates generally to the fields of molecular biology and therapeutic diagnosis. More particularly, it concerns methods and compositions involving prognosing, diagnosing, and treating hyperproliferative disorders.

### II. Background

Hyperproliferative disorders such as ovarian cancer and endometriosis are particularly problematic to detect and diagnose. Endometriosis affects 6-11% of premenopausal women, causing pain, infertility, reduced quality of life and billions of $70 Billion in 2016 (GlobalData 2018) in health care related costs annually. Although, the diagnosed prevalence of endometriosis is lower than other chronic diseases, the economic burden of endometriosis is comparable to other chronic diseases such as diabetes, Crohn's disease, and rheumatoid arthritis (RA). With a poorly understood etiology, endometriosis is characterized by hormonally responsive endometrial implants outside the uterus. In the case of ovarian cancer, this cancer often goes undetected until it has spread within the pelvis and abdomen.

Currently laparoscopic surgery is the only means for providing a definitive diagnosis of endometriosis and most patients struggle to find an effective treatment modality to relieve symptoms. While numerous studies have investigated non-invasive biomarkers for diagnosing endometriosis and predict prognosis and appropriate treatment, none have shown sufficient performance to be translated into clinical use. Endometriosis is also associated with non-modifiable risk factors such as family history, early age at menarche, and lower parity. Early identification of symptoms associated with endometriosis among high-risk women can limit the cost associated with progression of disease, and reduce the number of late-stage endometriosis cases. However, early diagnosis of endometriosis can be difficult as symptoms are often non-specific, there are often limited clinical signs on examination, laboratory testing is not helpful, and imaging is of only limited benefit. Hence, laparoscopy is required to make a definitive diagnosis. Due to these reasons, a delay of 7 (seven) to 12 (twelve) years on average between the development of symptoms and diagnosis of endometriosis occurs, which significantly impacts patients' quality-of-life.

The key challenge is early and precise diagnosis. Thus, there is a need for a rapid non-invasive diagnostic (RNID) that may help those women through proper and rapid diagnosis of their hyperproliferative disorder.

### SUMMARY

The current disclosure fulfills the aforementioned need in the by providing sensitive diagnostic methods and kits that do not require invasive laparoscopic surgery. It was found that monomeric myeloperoxidase (MPO) was found in cells, tissues, and the sera of patients with endometriosis. This novel biomarker can be used to detect ovarian and endometrial hyperproliferative disorders. Accordingly, aspects of the disclosure relate to a method for evaluating a subject comprising detecting monomeric myeloperoxidase (MPO) in a biological sample from the subject. Also disclosed is a method for treating a subject with an endometrial or ovarian hyperproliferative disorder, the method comprising administering an treatment for the endometrial or ovarian hyperproliferative disorder to a subject that has had the level of monomeric MPO evaluated in a biological sample from the subject.

Further aspects of the disclosure relate to a method of diagnosing a subject with an endometrial or ovarian hyperproliferative disorder comprising: a) evaluating monomeric MPO in a biological sample from the subject; b) comparing the measured level to control level or control samples; and c) diagnosing the subject with an endometrial or ovarian hyperproliferative disorder based on the measured level of monomeric MPO. Further aspects relate to a method for monitoring a subject being treated for an endometrial or ovarian hyperproliferative disorder with a therapeutic agent, the method comprising a) evaluating monomeric MPO in a biological sample from the subject; b) comparing the measured level to control level or control samples; and c) determining the efficacy of the therapeutic agent based on the measured level of monomeric MPO. Further aspects relate to a method for making a complex comprising contacting a biological sample with an antibody that binds to monomeric MPO.

Further aspects relate to a kit comprising one or more anti-MPO antibodies or an MPO binding fragment thereof and a size exclusion column that fractionates a sample and wherein the fractionation separates polypeptides having a size of 75 kDa and polypeptide having a size of 150 kDa into separate fractions.

Methods of the disclosure include prognosing, diagnosing, or monitoring subject having an endometrial or ovarian hyperproliferative disorder, suspected of having endometrial or ovarian hyperproliferative disorder, and/or having symptoms of endometrial or ovarian hyperproliferative disorder. The subject may be diagnosed as having a stage I, II, III, or IV cancer based on the evaluated level of monomeric MPO.

The term hyperproliferative disorder refers to a disorder that in characterized by an abnormal growth or hyperproliferation of tissue. The hyperproliferative disorder may include hyperplasia, dysplasia, and pre-cancerous lesions. The hyperproliferation may be further characterized by a tumor. In some aspects the hyperproliferation is benign, meaning that it is non-cancerous. In some aspects, the hyperproliferation is malignant, which refers to a cancerous growth.

The biological sample may comprise serum, plasma, or tissue samples. In some aspects, the biological sample is a blood sample or a fraction thereof. In some aspects, the biological sample is a biological sample described herein. In certain aspects, the biological sample is a serum sample. In some aspects, the biological sample is a plasma sample.

Aspects of the disclosure also relate to evaluating iron levels in a biological sample from the subject. The evaluation of the iron level may be combined with the evaluation of monomeric MPO to provide a diagnosis or prognosis for the subject. In some aspects, the method further comprises detecting iron levels in the biological sample from the subject. In some aspects, detecting iron levels in the biological sample from the subject comprises one or more of a serum iron test, free iron test, a total iron-binding capacity (TIBC) test, or a ferritin test. In method aspects of the disclosure, the subject is one that has been evaluated for iron levels. In some aspects, the subject has been evaluated for iron levels by having one or more of a serum iron test, a total iron-binding capacity (TIBC) test, or a ferritin test in a biological sample from the subject. In some aspects, the subject has been determined to have abnormal iron levels. In some aspects, the subject has been determined to a lower than normal elevated TIBC. In some aspects, the subject has been determined to have elevated ferritin. In some aspects, the subject has been determined to have elevated serum iron. In some aspects, the subject has been determined to have normal iron levels. The normal range for TIBC is 250 to 450 mcg/dL. In some aspects, the subject is determined to have a TIBC of less than 250 mcg/dL. The normal range for ferritin is: 20 to 250 ng/mL for adult males, 10 to 120 ng/mL for adult females, 18 to 39 years, and 12 to 263 ng/ml for females 40 years and older. In some aspects, the ferritin is determined to be greater than 120 ng/ml, 250 ng/ml, or 263 ng/ml. In some aspects, the subject is determined to have abnormal transferrin saturation. In some aspects, the subject is determined to have elevated transferrin saturation. Normal ranges for transferrin saturation is 20-50%. In some aspects, the subject is determined to have or the biological sample has greater than 50% transferrin saturation. In some aspects, the subject has been determined to have, has, or the biological sample from the subject has elevated serum iron levels. The normal range for serum iron is 60 to 170 micrograms per deciliter (mcg/dL), or 10.74 to 30.43 micromoles per liter (micromol/L). In some aspects, the subject has been determined to have, or the biological sample has a serum iron level of greater than 170 mcg/dL.

In some aspects, the TIBC in the biological sample is, the TIBC in the subject has been determined to be, or the TIBC in a biological sample from the subject was determined to be great than, less than, at least, or at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, or 550 mcg/dL, or any derivable range therein. In some aspects, the ferritin in the biological sample is, the ferritin in the subject has been determined to be, or the ferritin in a biological sample from the subject was determined to be great than, less than, at least, or at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or 350 ng/mL, or any range derivable therein. In some aspects, the transferrin saturation in the biological sample is, the transferrin saturation in the subject has been determined to be, or the transferrin saturation in a biological sample from the subject was determined to be great than, less than, at least, or at most 5, 10, 15, 20, 25, 30, 35, 40, 45, 50, 55, 60, 65, 70, 75, 80, 85, 90, or 95% (or any derivable range therein). In some aspects, the transferrin saturation in the biological sample is, the transferrin saturation in the subject has been determined to be, or the transferrin saturation in a biological sample from the subject was determined to be great than, less than, at least, or at most 30, 40, 50, 60, 70, 80, 90, 100, 110, 120, 130, 140, 150, 160, 170, 180, 190, 200, 210, 220, 230, 240, 250, 260, or 270 micrograms per deciliter, or any derivable range therein. In some aspects, the free iron in the biological sample is, the free iron in the subject has been determined to be, or the free iron in a biological sample from the subject was determined to be great than, less than, at least, or at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, or 550 mcg/dL, or any derivable range therein. In some aspects, the higher than normal level of free iron in the biological sample from the subject is greater than 100, greater than 110, greater than 120, greater than 130, greater than 140, greater than 150 mcg/dL, or any derivable range therein.

The methods of the disclosure may comprise or further comprise size fractionation of the biological sample. In some aspects, the biological sample is a size-fractionated sample. The size fractionation in the methods of the disclosure may be performed in a manner that provides a fraction of the biological sample that comprises molecules that are 75 kDa and excludes molecules that are 150 kDa or larger. In some aspects, the biological sample is a size-fractionated sample that comprises molecules that are 75 kDa and excludes molecules that are 150 kDa or larger. The size fractionation may be one that separates the sample into a fraction comprising molecules that are in a size range of 10-100 kDa, 30-100 kDa, 40-100 kDa, 50-100 kDa, 60-90 kDa, or 60-85 kDa. In some aspects, the size fractionation may provide for a fraction that is in the range of about 10, 15, 20, 25, 30, 35, 40, 45, 50, 55, 60, 65, or 70 kDa to about 80, 85, 90, 95, 100, 105, 110, 115, 120, 125, 130, 135, 140, or 145 kDa (and all derivable ranges therein). In some aspects, the average size of the fraction of the biological sample is a size of, a size of at least, or a size of at most 10, 15, 20, 25, 30, 35, 40, 45, 50, 55, 60, 65, 70, 75, 80, 5, 90, 95, 100, 105, 110, 115, 120, 125, 130, 135, 140, 145, 150, 155, 160, 165, 170, 175, 180, 185, or 190 kDa (or any range derivable therein). In some aspects, the fraction excludes molecules that are, that are at least, or that are at most 10, 15, 20, 25, 30, 35, 40, 45, 50, 55, 60, 65, 70, 75, 80, 5, 90, 95, 100, 105, 110, 115, 120, 125, 130, 135, 140, 145, 150, 155, 160, 165, 170, 175, 180, 185, or 190 kDa (or any range derivable therein). In some aspects, the method comprises detecting monomeric MPO in the fraction comprising molecules that are 75 kDa.

In aspects of the disclosure, detecting monomeric MPO comprises immunological detection of monomeric MPO. In some aspects, detecting or evaluating monomeric MPO comprises an enzyme-linked immunosorbent assay (ELISA) assay. An Elisa assay uses a solid-phase type of enzyme immunoassay (EIA) to detect the presence of a ligand (commonly a protein) in a liquid sample using antibodies directed against the protein to be measured. In some aspects, antigens from the biological sample or fraction thereof are attached to a surface. Then, an antibody, such as an anti-MPO antibody, may be applied over the surface so it can bind to any MPO from the biological sample. This antibody may be linked to a detection molecule, such as an enzyme, and then any unbound antibodies may be removed. In the final step, the detection molecule may be detected qualitatively or quantitatively. In the aspect in which the detection molecule is an enzyme, the enzyme's substrate may be added, leading to a reaction that produces a detectable signal, most commonly a color change that can be quantitatively or qualitatively measured.

In some aspects, the ELISA is further characterized as a sandwich ELISA. An anti-MPO antibody may be immobilized on a solid support, such as a microtiter plate or a polystyrene microtiter plate. The biological sample or fraction may be added to the solid support to allow binding between the anti-MPO antibody and MPO in the biological sample or fraction. Unbound molecules may be washed away from the solid support. After the MPO antigen is immobilized, the detection antibody can be added, forming a complex with the antigen. The detection antibody can be covalently linked to a detection molecule, such as an enzyme or can itself be detected by a secondary antibody that is linked to a detection molecule, such as an enzyme. Between each step, the plate is typically washed with a mild detergent solution to remove any proteins or antibodies that are non-specifically bound. After the final wash step, the plate is developed by qualitatively or quantitatively detecting the detection molecule. In the case of enzymatic detection, the final step may comprise adding an enzymatic substrate to produce a visible signal that can qualitatively or quantitatively detected.

The ELISA may be performed using other forms of ligand binding assays instead of strictly immunoassays. An ELISA may be one that comprises any ligating reagent that can be immobilized on the solid phase along with a detection reagent that will bind specifically and use a detectable molecule to generate a signal that can be properly quantified. In between the washes, only the ligand and its specific binding counterparts remain specifically bound or “immunosorbed” by antigen-antibody interactions to the solid phase, while the nonspecific or unbound components are washed away.

“Detectable labels, molecules, or moieties” or “detection molecules, labels, or moieties” are used interchangeably and refer to compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275, 149 and 4,366,241; each incorporated herein by reference. Molecules containing azido groups may also be used to form covalent bonds to proteins through reactive nitrene intermediates that are generated by low intensity ultraviolet light (Potter & Haley, 1983).

In some aspects, detecting monomeric MPO comprises contacting the biological sample or the fraction comprising molecules that are 75 kDa with an anti-MPO antibody or MPO binding molecule under conditions that allow for the binding of MPO to the anti-MPO antibody. In some aspects, the anti-MPO antibody or binding molecule is linked to a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The methods of the disclosure may comprise or further comprise washing the solid support to remove unbound molecules. In some aspects, the methods comprise or further comprise contacting the biological sample or the fraction with a capture antibody or antigen-binding molecule. In some aspects, the capture antibody or antigen-binding molecule comprise a second anti-MPO antibody or MPO antigen-binding fragment. Exemplary antigen-binding fragments include, for example, a single chain variable fragment (scFv), F(ab′)₂, Fab′, Fab, Fv, or rIgG. In some aspects, the capture antibody is linked to a detectable label. The method may comprise or further comprise quantitatively or qualitatively evaluating the detectable label.

In aspects of the disclosure, the subject or patient may be a human subject or a human patient. In some aspects, the subject or patient is a non-human animal. In some aspects, the non-human animal is a bat, monkey, camel, rat, mouse, rabbit, goat, chicken, bird, cat, dog, The subject may further be defined as a high risk subject. In some aspects, the subject is one that has one or more symptoms of endometriosis and/or ovarian cancer. Symptoms of ovarian cancer include abdominal bloating or swelling, quickly feeling full when eating, weight loss, discomfort in the pelvis area, changes in bowel habits, such as constipation, and a frequent need to urinate. The subject may also be one that has been diagnosed with an endometrial or ovarian hyperproliferative disorder. In some aspects, the subject has been treated for an endometrial or ovarian hyperproliferative disorder. In some aspects, the subject, will be treated for an endometrial or ovarian hyperproliferative disorder. In some aspects, the subject is currently undergoing treatment for an endometrial or ovarian hyperproliferative disorder. The subject may be further defined as a human subject. In some aspects, the subject is female. In some aspects, the subject is on hormone therapy. The hormone therapy may comprise contraception or hormone replacement therapy. In some aspects, the female subject is an adolescent, perimenopausal, or menopausal female. In some aspects, the subject is one that has one or more symptoms of endometrial or ovarian hyperproliferative disorders.

The method may comprise or further comprise quantitating the level of monomeric MPO in the biological sample. In some aspects, the level of monomeric MPO in the biological sample or fraction has been quantitated. In some aspects, the level of monomeric MPO is normalized. In some aspects, the level of monomeric MPO is compared to a control. In some aspects, the level of monomeric MPO is determined to be greater than the control. In some aspects, the level of monomeric MPO is determined to be less than the control. In some aspects, the subject has or has been determined to have a level of monomeric MPO in the biological sample that is greater than the level of a monomeric MPO in a control sample. In some aspects, has or has been determined to have a level of monomeric MPO in the biological sample that is less than the level of a monomeric MPO in a control sample. In some aspects, the subject has or has been determined to have a level of monomeric MPO in the biological sample that is not significantly different than the level of a monomeric MPO in a control sample. For example, the level of monomeric MPO may be determined to be, to be at least, or to be at most 0.5, 1, 1.5, 2, 2.5, 3, 3.5, 4, or 5 standard deviations different than or within a control value. In some aspects, the level of monomeric MPO may be determined to be, to be at least, or to be at most 1, 2, 5, 10, 15, 20, 25, 30, 35, 40, 45, 50, 55, 60, 65, 70, 75, 80, 85, 90, 95, 98, 99, or 100% (or any derivable range therein) above or below a control level of monomeric MPO. The control may comprise the level of monomeric MPO that is representative of the level of monomeric MPO in a biological sample from a subject with endometriosis. In some instances, the control may comprise the level of monomeric MPO that is representative of the level of monomeric MPO in a biological sample from a subject with ovarian cancer. In some aspects, the control may comprise the level of monomeric MPO that is representative of the level of monomeric MPO in a biological sample from a subject without an endometrial or ovarian hyperproliferative disorders. In some aspects, the method further comprises diagnosing the subject. In some aspects, the subject is diagnosed with ovarian cancer based on the determined level of monomeric MPO. In some aspects, the subject is diagnosed with or the cancer comprises stage I, II, III, or IV ovarian cancer based on the determined level of monomeric MPO. The methods of the disclosure may also comprise or further comprise treating the subject for ovarian cancer. In some aspects, the subject is diagnosed with endometriosis based on the determined level of MPO. The methods of the disclosure may comprise or further comprise treating the subject for endometriosis.

In some aspects, the therapeutic agent is determined to be effective when monomeric MPO: i) is not detected in the biological sample from the subject; ii) is not significantly different than a control, wherein the control comprises the level of monomeric MPO that is representative of the level of monomeric MPO in a biological sample from a subject without an endometrial or ovarian hyperproliferative disorders; iii) is less than a control, wherein the control comprises the level of monomeric MPO that is representative of the level of monomeric MPO in a biological sample from a subject with an endometrial or ovarian hyperproliferative disorder; or iv) is decreased compared to the level of monomeric MPO before treatment of the subject with the therapeutic agent. In some aspects, the therapeutic agent is determined to be ineffective when monomeric MPO: i) detected in the biological sample from the subject; ii) is increased compared to a control, wherein the control comprises the level of monomeric MPO that is representative of the level of monomeric MPO in a biological sample from a subject without an endometrial or ovarian hyperproliferative disorders; iii) is not significantly different or more than a control, wherein the control comprises the level of monomeric MPO that is representative of the level of monomeric MPO in a biological sample from a subject with an endometrial or ovarian hyperproliferative disorder; or iv) is not significantly different or increased compared to the level of monomeric MPO before treatment of the subject with the therapeutic agent.

In some aspects, the method further comprises evaluating the level of monomeric MPO in a biological sample from the subject obtained prior to treatment. In some aspects, the method further comprises evaluating the level of monomeric MPO in a biological sample from the subject obtained after one or more treatments. For example, the method may comprise or further comprise evaluating monomeric MPO levels after one dose, after two doses, after three doses, after four doses, after five doses, and/or after six doses of a particular treatment. Treatment efficacy may be determined based on the evaluated levels of monomeric MPO.

In some aspects, the level of monomeric MPO is determined to be, determined to be at least, or determined to be at most 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 325, 350, 375, 400, 425, 450, 475, or 500 ng/ml, mcg/ml, mg/ml, (or any derivable range therein) in the biological sample from the subject. In some aspects, the subject is diagnosed with Stage I cancer when the level of monomeric MPO is determined to be, determined to be at least, or determined to be at most 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 325, 350, 375, 400, 425, 450, 475, or 500 ng/ml, mcg/ml, mg/ml, (or any derivable range therein) in the biological sample from the subject. In some aspects, the subject is diagnosed with Stage II cancer when the level of monomeric MPO is determined to be, determined to be at least, or determined to be at most 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 325, 350, 375, 400, 425, 450, 475, or 500 ng/ml, mcg/ml, mg/ml, (or any derivable range therein) in the biological sample from the subject. In some aspects, the subject is diagnosed with Stage III cancer when the level of monomeric MPO is determined to be, determined to be at least, or determined to be at most 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 325, 350, 375, 400, 425, 450, 475, or 500 ng/ml, mcg/ml, mg/ml, (or any derivable range therein) in the biological sample from the subject. In some aspects, the subject is diagnosed with Stage IV cancer when the level of monomeric MPO is determined to be, determined to be at least, or determined to be at most 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 325, 350, 375, 400, 425, 450, 475, or 500 ng/ml, mcg/ml, mg/ml, (or any derivable range therein) in the biological sample from the subject. In aspects, of the disclosure, late stage cancer comprises Stage IV and/or Stage III. In aspects of the disclosure, early stage cancer comprises Stage I and/or Stage II.

The control level may comprise the level of monomeric MPO that is representative of the level of monomeric MPO in a subject with endometriosis. In some aspects, the control comprises the level of monomeric MPO that is representative of the level of monomeric MPO in a subject with ovarian cancer. In some aspects, the control comprises the level of monomeric MPO that is representative of the level of monomeric MPO in a subject without an endometrial or ovarian hyperproliferative disorders. In some aspects, the hyperproliferative disorder comprises endometriosis. In some aspects, the hyperproliferative disorder comprises ovarian cancer.

The treatment may be one known in the art for endometriosis or ovarian cancer or one described herein. In some aspects, the treatment comprises hormone therapy, or surgery. Other treatments useful in the methods of the disclosure include surgery, radiation, chemotherapy, hormone therapy, immunotherapy, or targeted therapy.

In some aspects, the subject is diagnosed as not having an endometrial or ovarian hyperproliferative disorder when monomeric MPO: i) is not detected in the biological sample from the subject; ii) is not significantly different than a control, wherein the control comprises the level of monomeric MPO that is representative of the level of monomeric MPO in a biological sample from a subject without an endometrial or ovarian hyperproliferative disorders; or iii) is less than a control, wherein the control comprises the level of monomeric MPO that is representative of the level of monomeric MPO in a biological sample from a subject with an endometrial or ovarian hyperproliferative disorder. In some aspects, the subject is diagnosed as having endometriosis when monomeric MPO: i) is greater than a control, wherein the control comprises the level of monomeric MPO that is representative of the level of monomeric MPO in a biological sample from a subject without an endometrial or ovarian hyperproliferative disorders; ii) is not significantly different than the control; wherein the control comprises the level of monomeric MPO that is representative of the level of monomeric MPO in a biological sample from a subject with endometriosis; or iii) is less than a control, wherein the control comprises the level of monomeric MPO that is representative of the level of monomeric MPO in a biological sample from a subject with ovarian cancer. In some aspects, the subject is diagnosed as having ovarian cancer when monomeric MPO: i) is greater than a control, wherein the control comprises the level of monomeric MPO that is representative of the level of monomeric MPO in a biological sample from a subject without an endometrial or ovarian hyperproliferative disorders or from a subject with endometriosis; ii) is not significantly different than a control, wherein the control comprises the level of monomeric MPO that is representative of the level of monomeric MPO in a biological sample from a subject with ovarian cancer. In some aspects, evaluating monomeric MPO comprises qualitatively detecting the level of monomeric MPO in the biological sample. In some aspects, the subject is diagnosed as not having an endometrial or ovarian hyperproliferative disorder when monomeric MPO in not detected in the biological sample from the subject. In some aspects, the subject is diagnosed as having an endometrial or ovarian hyperproliferative disorder when monomeric MPO is detected in the biological sample from the subject.

Kit aspects of the disclosure may comprise or further comprise one or more negative or positive control samples. In some aspects, the kit comprises an ELISA for detecting MPO. In some aspects, the anti-MPO antibody or binding fragment is operatively linked to a solid support. In some aspects, the kit comprises at least two anti-MPO antibodies, at least two anti-MPO antibody binding fragments, or one anti-MPO antibody and one anti-MPO antibody binding fragment. In some aspects, the one or more anti-MPO antibodies or MPO antibody binding fragments is linked to a detectable label.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments or aspects discussed elsewhere in a different Example or elsewhere in the application, such as in the Summary of Invention, Detailed Description of the Embodiments, Claims, and description of Figure Legends.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment or aspect discussed with respect to one aspect of the disclosure applies to other aspects and embodiments of the disclosure as well and vice versa.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: A representative image of monomeric and dimeric forms of MPO in Endometriosis. Western blot analysis was performed utilizing non-reducing gels to detect monomeric and dimeric forms of MPO. Only the monomeric form of MPO is detected in endometriosis tissues as evident by the 75 kDa band.

FIG. 2: A representative image of monomeric and dimeric forms of MPO in ovarian cancer. Western blot analysis was performed utilizing non-reducing gels to detect monomeric and dimeric forms of MPO in serum from healthy volunteer, serum from an ovarian cancer patient, and selected ovarian cancer cell lines. Only the monomeric form of MPO is detected in ovarian cancer cell lines as evident by the 75 kDa band. Intriguingly, the monomeric form of MPO is also detected in serum obtained from ovarian cancer patients but not in healthy serum which has only shown the dimeric MPO form as depicted by the 150 kDa band.

FIG. 3: Real time RT-PCR analysis of MPO in multiple cancer cell lines. MPO mRNA level was determined in epithelial ovarian cancer (SKOV-3, MDAH-2774, OVCAR-3, OV-21, OV-90), pancreatic cancer (BXPC-3), colon cancer (COLO-3), non-Hodgkin's B cell lymphoma (DLCL-2), bladder cancer (HTB-4), and endometrial cancer (CRL-1671) cell lines.

FIG. 4: Boxplot showing dimer/monomer serum MPO concentrations in four different conditions [Control=Healthy controls (n=15), NC_IGC=Non-Cancer Inflammatory Gynecological Disease (n=14), ES_EOC=Early Stage Epithelial Ovarian Cancer (n=12), LS_EOC=Late Stage Epithelial Ovarian Cancer (n=16)], where x-axis is log 10 scaled.

FIG. 5: Receiver Operating Curves, and area under the curve (AUC) comparing the predictive power of serum dimer MPO 84.5% and monomer MPO 100% in discriminating samples between NC_IGC=Non-Cancer Inflammatory Gynecological Disease and ES_EOC=Early Stage Epithelial Ovarian cancer (p=0.050) and between ES_EOC=Early Stage Epithelial Ovarian Cancer and LS_EOC=Late Stage Epithelial Ovarian Cancer (p=0.05).

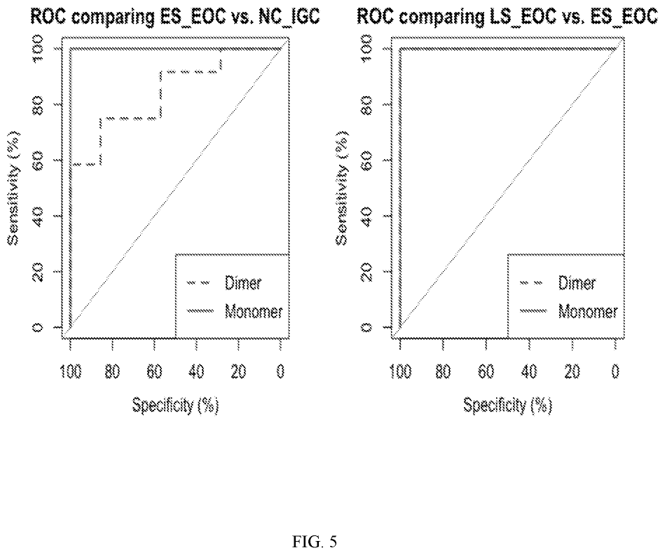

### DETAILED DESCRIPTION OF THE INVENTION

Here, the inventors report that ovarian cancer cells express only the monomer form of MPO which discriminated between early and late stage of the disease. Total RNA and proteins were isolated from various human ovarian cancer cells, sera from ovarian cancer patients with various stages, sera from non-cancer inflammatory gynecological disease, and healthy volunteers. A combination of ELISA and non-reducing western blot were used to determine levels of monomeric and dimeric MPO in all samples. Real time PCR was utilized to measure MPO mRNA levels in various cancer cell lines. Receiver operating curves (ROC) were used to compare the predictive power of serum dimeric and monomeric MPO and discriminating between samples. The inventors demonstrated that monomeric MPO is the predominant form of MPO in ovarian cancer cell lines. Intriguingly, monomeric MPO is also detected in serum obtained from an ovarian cancer patient with various stages but not in healthy individuals. More importantly, monomeric MPO discriminated between early stage and late stage of the disease. The novel finding from this study emphasizes MPO as a biomarker that is critically needed for early detection of this disease.

### II. Hyperproliferative Disorder Treatments

Methods and compositions may be provided for treating hyperproliferative disorders with particular applications of biomarker expression or activity levels. Based on a profile of biomarker expression or activity levels, different treatments may be prescribed or recommended for different patients. In some aspects, the hyperproliferative disorder comprises ovarian cancer or a cancer originating in the ovarian epithelium, germ cells, or stroma, fallopian epithelium, ovaries, cervix, fallopian tube, or uterus. In some aspects, the hyperproliferative disorder is primary peritoneal carcinoma, fallopian tube cancer, teratoma, dysgerminoma, or a yolk sac tumor. The cancer may comprise a cancer stage, TNM, and/or is further characterized as having features described below.

### A. Cancer Staging

The most common staging system is the TNM (for tumors/nodes/metastases) system, from the American Joint Committee on Cancer (AJCC). The TNM system assigns a number based on three categories. “T” denotes the degree of invasion of the intestinal wall, “N” the degree of lymphatic node involvement, and “M” the degree of metastasis. The broader stage of a cancer is usually quoted as a number I, II, III, IV derived from the TNM value grouped by prognosis; a higher number indicates a more advanced cancer and likely a worse outcome. Details of this system are in the tables below:

| AJCC/ | Stage | |
|---|---|---|
| FIGO stage | Grouping | Stage Description* |
| I | T1 | The cancer is only in the ovary (or ovaries) or fallopian tube(s) (T1). |
| | N0 | It has not spread to nearby lymph nodes (N0) or to distant sites (M0). |
| | M0 | |
| IA | T1a | The cancer is in one ovary, and the tumor is confined to the inside of the ovary; |
| | N0 | or the cancer is in one fallopian tube, and is only inside the fallopian tube. |
| | M0 | There is no cancer on the outer surfaces of the ovary or fallopian tube. No |
| | | cancer cells are found in the fluid (ascites) or washings from the abdomen and |
| | | pelvis (T1a). |
| | | It has not spread to nearby lymph nodes (N0) or to distant sites (M0). |
| IB | T1b | The cancer is in both ovaries or fallopian tubes but not on their outer surfaces. |
| | N0 | No cancer cells are found in the fluid (ascites) or washings from the abdomen |
| | M0 | and pelvis (T1b). It has not spread to nearby lymph nodes (N0) or to distant |
| | | sites (M0). |
| IC | T1c | The cancer is in one or both ovaries or fallopian tubes and any of the following |
| | N0 | are present: |
| | M0 | The tissue (capsule) surrounding the tumor broke during surgery, |
| | | which could allow cancer cells to leak into the abdomen and pelvis |
| | | (called surgical spill). This is stage IC1. |
| | | Cancer is on the outer surface of at least one of the ovaries or fallopian |
| | | tubes or the capsule (tissue surrounding the tumor) has ruptured (burst) |
| | | before surgery (which could allow cancer cells to spill into the |
| | | abdomen and pelvis). This is stage IC2. |
| | | Cancer cells are found in the fluid (ascites) or washings from the |
| | | abdomen and pelvis. This is stage IC3. |
| | | It has not spread to nearby lymph nodes (N0) or to distant sites (M0). |
| II | T2 | The cancer is in one or both ovaries or fallopian tubes and has spread to other |
| | N0 | organs (such as the uterus, bladder, the sigmoid colon, or the rectum) within |
| | M0 | the pelvis or there is primary peritoneal cancer (T2). It has not spread to nearby |
| | | lymph nodes (N0) or to distant sites (M0). |
| IIA | T2a | The cancer has spread to or has invaded (grown into) the uterus or the fallopian |
| | N0 | tubes, or the ovaries. (T2a). It has not spread to nearby lymph nodes (N0) or to |
| | M0 | distant sites (M0). |
| IIB | T2b | The cancer is on the outer surface of or has grown into other nearby pelvic |
| | N0 | organs such as the bladder, the sigmoid colon, or the rectum (T2b). It has not |
| | M0 | spread to nearby lymph nodes (N0) or to distant sites (M0). |
| IIIA1 | T1 or T2 | The cancer is in one or both ovaries or fallopian tubes, or there is primary |
| | N1 | peritoneal cancer (T1) and it may have spread or grown into nearby organs in |
| | M0 | the pelvis (T2). It has spread to the retroperitoneal (pelvic and/or para-aortic) |
| | | lymph nodes only. It has not spread to distant sites (M0). |
| IIIA2 | T3a | The cancer is in one or both ovaries or fallopian tubes, or there is primary |
| | N0 or N1 | peritoneal cancer and it has spread or grown into organs outside the pelvis. |
| | M0 | During surgery, no cancer is visible in the abdomen (outside of the pelvis) to |
| | | the naked eye, but tiny deposits of cancer are found in the lining of the |
| | | abdomen when it is examined in the lab (T3a). |
| | | The cancer might or might not have spread to retroperitoneal lymph nodes (N0 |
| | | or N1), but it has not spread to distant sites (M0). |
| IIIB | T3b | There is cancer in one or both ovaries or fallopian tubes, or there is primary |
| | N0 or N1 | peritoneal cancer and it has spread or grown into organs outside the pelvis. The |
| | M0 | deposits of cancer are large enough for the surgeon to see, but are no bigger |
| | | than 2 cm (about ¾ inch) across. (T3b). |
| | | It may or may not have spread to the retroperitoneal lymph nodes (N0 or N1), |
| | | but it has not spread to the inside of the liver or spleen or to distant sites (M0). |
| IIIC | T3c | The cancer is in one or both ovaries or fallopian tubes, or there is primary |
| | N0 or N1 | peritoneal cancer and it has spread or grown into organs outside the pelvis. The |
| | M0 | deposits of cancer are larger than 2 cm (about ¾ inch) across and may be on |
| | | the outside (the capsule) of the liver or spleen (T3c). |
| | | It may or may not have spread to the retroperitoneal lymph nodes (N0 or N1), |
| | | but it has not spread to the inside of the liver or spleen or to distant sites (M0). |
| IVA | Any T | Cancer cells are found in the fluid around the lungs (called a malignant pleural |
| | Any N | effusion) with no other areas of cancer spread such as the liver, spleen, |
| | M1a | intestine, or lymph nodes outside the abdomen (M1a). |
| IVB | Any T | The cancer has spread to the inside of the spleen or liver, to lymph nodes other |
| | Any N | than the retroperitoneal lymph nodes, and/or to other organs or tissues outside |
| | M1b | the peritoneal cavity such as the lungs and bones (M1b). |

The “cancer” referred to in the methods described herein may include or exclude any of the above stages or TNM categories. The “cancer” referred to in the methods described herein may include or exclude any of the above stages or TNM categories. For example the cancer may be or may exclude Stage 0, I, IA, IB, IC, II, IIA, IIB, IIIA1, IIIA2, IIIB, IIIC, IVA, or IVB cancer. The patient may be one that has and/or has been determined to have Stage 0, I, IA, IB, IC, II, IIA, IIB, IIIA1, IIIA2, IIIB, IIIC, IVA, or IVB cancer. Furthermore, the cancer may be stage N0 and/or M0; T1, N0, and/or M0; T1, N1, and/or M0; T2, N0, and/or M0; T1, N2, and/or M0; T2, N1, and/or M0; T3, N0, and/or M0; T1, N3, and/or M0; T2, N2, and/or M0; T3, N1, and/or M0; T4a, N0, and/or M0; T2, N3, and/or M0; T3, N2, and/or M0; T4a, N1, and/or M0; T3, N3, and/or M0; T4a, N2, and/or M0; T4b and/or NO; N1 and/or M0; T4a, N3, and/or M0; T4b and/or N2; N3 and/or M0; Any T, any N, and/or M1.

### B. Treatments

Methods of the disclosure relate to treating subjects and patients with a cancer therapy. The cancer therapy may be one described below and may be given with respect to a patient having been determined to have a certain biomarker profile. For example, in some aspects, the therapy described below is given to a patient with a poor prognosis, unfavorable prognosis, or to a patient determined to be high risk. In some aspects, the therapy described below is given to a patient with a favorable prognosis, or to a patient determined to be low risk. Also contemplated are combinations of the therapies described below.

The cancer treatment may be surgery, radiation, chemotherapies, hormone therapies, or targeted therapies. The radiation may be further characterized as external beam radiation therapy or brachytherapy. The chemotherapy may be a platinum compound and/or a taxane. Platinum compounds include cisplatin and carboplatin. Taxanes include paclitaxel and docetaxel. In some aspects, the chemotherapy comprises a combination of a chemotherapeutic platinum compound and a taxane. Further chemotherapies include albumin bound paclitaxel, altretamine, capecitabine, cyclophosphamide, etoposide, gemcitabine, ifosfamide, irinotecan, liposomal doxorubicin, melphalan, pemetrexed, topotecan, and vinorelbine.

Common ways to give chemotherapy include an intravenous (IV) tube placed into a vein using a needle or in a pill or capsule that is swallowed (orally). A chemotherapy regimen usually comprises a specific number of cycles given over a set period of time. A patient may receive 1 drug at a time or combinations of different drugs at the same time.

Antimetabolites can be used in cancer treatment, as they interfere with DNA production and therefore cell division and the growth of tumors. Because cancer cells spend more time dividing than other cells, inhibiting cell division harms tumor cells more than other cells. Anti-metabolites masquerade as a purine (azathioprine, mercaptopurine) or a pyrimidine, chemicals that become the building-blocks of DNA. They prevent these substances becoming incorporated in to DNA during the S phase (of the cell cycle), stopping normal development and division. They also affect RNA synthesis. However, because thymidine is used in DNA but not in RNA (where uracil is used instead), inhibition of thymidine synthesis via thymidylate synthase selectively inhibits DNA synthesis over RNA synthesis. Due to their efficiency, these drugs are the most widely used cytostatics. In the ATC system, they are classified under L01B.

Thymidylate synthase inhibitors are chemical agents which inhibit the enzyme thymidylate synthase and have potential as an anticancer chemotherapy. As an anti-cancer chemotherapy target, thymidylate synthetase can be inhibited by the thymidylate synthase inhibitors such as fluorinated pyrimidine fluorouracil, or certain folate analogues, the most notable one being raltitrexed (trade name Tomudex). Additional agents include pemetrexed, nolatrexed, ZD9331, and GS7904L.

In further aspects, there may be involved prodrugs that can be converted to thymidylate synthase inhibitors in the body, such as Capecitabine (INN), an orally administered chemotherapeutic agent used in the treatment of numerous cancers. Capecitabine is a prodrug, that is enzymatically converted to 5-fluorouracil in the body.

If cancer has entered the lymph nodes, adding the chemotherapy agents fluorouracil or capecitabine increases life expectancy. Chemotherapy agents for this condition may include capecitabine, fluorouracil, irinotecan, leucovorin, oxaliplatin and UFT. Another type of agent that is sometimes used are the epidermal growth factor receptor inhibitors.

In certain aspects, alternative treatments may be prescribed or recommended based on the biomarker profile. In addition to traditional chemotherapy for gastric cancer patients, cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

Just as for chemotherapy, radiotherapy can be used in the neoadjuvant and adjuvant setting for some stages of cancer.

Targeted therapy may also be used in the methods described herein. Targeted therapies include angiogenesis inhibitors such as bevacizumab and/or PARP inhibitors such as, olaparib, rucaparib, and/or niraparib. Also included are NTRK targeted drugs, such as Larotrectinib and entrectinib.

Hormone therapies include luteinizing-hormone-releasing agonists, tamoxifen, and aromatase inhibitors.

Immunotherapies that are designed to boost the body's natural defenses to fight the cancer may also be used. Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Generally, the tumor cell must bear some marker that is amenable to targeting, i.e., is not present on the majority of other cells. Many tumor markers exist and any of these may be suitable for targeting.

### C. Monitoring

In certain aspects, the biomarker-based method may be combined with one or more other cancer diagnosis or screening tests at increased frequency if the patient is determined to be at high risk for recurrence or have a poor prognosis based on the biomarker as described above.

In some aspects, the methods of the disclosure further include one or more monitoring tests. The monitoring protocol may include any methods known in the art. In particular,

the monitoring include obtaining a sample and testing the sample for diagnosis. For example, the monitoring may include endoscopy, biopsy, laparoscopy, colonoscopy, blood test, genetic testing, endoscopic ultrasound, X-ray, barium enema x-ray, chest x-ray, barium swallow, a CT scan, a MRI, a PET scan, or HER2 testing. In some aspects, the monitoring test comprises radiographic imaging. Examples of radiographic imaging this is useful in the methods of the disclosure includes hepatic ultrasound, computed tomographic (CT) abdominal scan, liver magnetic resonance imaging (MRI), body CT scan, and body MRI.

### D. ROC Analysis

In statistics, a receiver operating characteristic (ROC), or ROC curve, is a graphical plot that illustrates the performance of a binary classifier system as its discrimination threshold is varied. ROC analysis may be applied to determine a cut-off value or threshold setting of biomarker expression. For example, patients with biological samples determined to have biomarker expression value above a certain cut-off threshold but below a higher cut-off threshold may be determined to have endometriosis. Patients with biological samples determined to have a biomarker expression level that surpasses the cut-off threshold for endometriosis may be determined to have cancer. The curve is created by plotting the true positive rate against the false positive rate at various threshold settings. (The true-positive rate is also known as sensitivity in biomedical informatics, or recall in machine learning. The false-positive rate is also known as the fall-out and can be calculated as 1−specificity). The ROC curve is thus the sensitivity as a function of fall-out. In general, if the probability distributions for both detection and false alarm are known, the ROC curve can be generated by plotting the cumulative distribution function (area under the probability distribution from −infinity to +infinity) of the detection probability in the y-axis versus the cumulative distribution function of the false-alarm probability in x-axis.

ROC analysis provides tools to select possibly optimal models and to discard suboptimal ones independently from (and prior to specifying) the cost context or the class distribution. ROC analysis is related in a direct and natural way to cost/benefit analysis of diagnostic decision making.

The ROC curve was first developed by electrical engineers and radar engineers during World War II for detecting enemy objects in battlefields and was soon introduced to psychology to account for perceptual detection of stimuli. ROC analysis since then has been used in medicine, radiology, biometrics, and other areas for many decades and is increasingly used in machine learning and data mining research.

The ROC is also known as a relative operating characteristic curve, because it is a comparison of two operating characteristics (TPR and FPR) as the criterion changes. ROC analysis curves are known in the art and described in Metz C E (1978) Basic principles of ROC analysis. Seminars in Nuclear Medicine 8:283-298; Youden W J (1950) An index for rating diagnostic tests. Cancer 3:32-35; Zweig M H, Campbell G (1993) Receiver-operating characteristic (ROC) plots: a fundamental evaluation tool in clinical medicine. Clinical Chemistry 39:561-577; and Greiner M, Pfeiffer D, Smith R D (2000) Principles and practical application of the receiver-operating characteristic analysis for diagnostic tests. Preventive Veterinary Medicine 45:23-41, which are herein incorporated by reference in their entirety. A ROC analysis may be used to create cut-off values for prognosis and/or diagnosis purposes.

### III. Protein Assays

A variety of techniques can be employed to measure expression levels of polypeptides and proteins in a biological sample to determine biomarker expression levels. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining protein expression levels of biomarkers.

In one aspect, antibodies, or antibody fragments or derivatives, can be used in methods such as Western blots, ELISA, or immunofluorescence techniques to detect biomarker expression. In some aspects, either the antibodies or proteins are immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of biomarkers. In some aspects, antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horseradish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art. A two-site, monoclonal-based immunoassay utilizing antibodies reactive to two non-interfering epitopes or a competitive binding assay may be employed.

Numerous labels are available and commonly known in the art. Radioisotope labels include, for example, 36S, 14C, 125I, 3H, and 131I. The antibody can be labeled with the radioisotope using the techniques known in the art. Fluorescent labels include, for example, labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques known in the art. Fluorescence can be quantified using a fluorimeter. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzymology (Ed. J. Langone & H. Van Vunakis), Academic press, New York, 73: 147-166 (1981).

In some aspects, a detection label is indirectly conjugated with an antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). In some aspects, the antibody need not be labeled, and the presence thereof can be detected using a labeled antibody, which binds to the antibody.

### IV. Sample Preparation

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain aspects the sample is obtained from a biopsy from ovarian or endometrial tissue by any of the biopsy methods previously mentioned. In other aspects the sample may be obtained from any of the tissues provided herein that include but are not limited to non-cancerous or cancerous tissue and non-cancerous or cancerous tissue from the ovarian epithelium, fallopian epithelium, ovaries, cervix, fallopian tube, or uterus. Alternatively, the sample may be obtained from any other source including but not limited to blood, serum, plasma, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple plasma or serum samples may be obtained for diagnosis by the methods described herein. In other cases, multiple samples, such as one or more samples from one tissue type (for example ovaries or related tissues) and one or more samples from another specimen (for example serum) may be obtained for diagnosis by the methods. Samples may be obtained at different times are stored and/or analyzed by different methods. For example, a sample may be obtained and analyzed by routine staining methods or any other cytological analysis methods.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, blood draw, endoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### V. Administration of Therapeutic Compositions

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some aspects, the first and second cancer treatments are administered in a separate composition. In some aspects, the first and second cancer treatments are in the same composition.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the cancer therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

### VI. Pharmaceutical Compositions

In certain aspects, the compositions or agents for use in the methods, such as chemotherapeutic agents or biomarker modulators, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body, such as skeletal muscle or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Topical administration may be particularly advantageous for the treatment of skin cancers, to prevent chemotherapy-induced alopecia or other dermal hyperproliferative disorder. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol is between about 0.01 ml and 0.5 ml.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### VII. Kits

Certain aspects of the present invention also concern kits containing compositions of the invention or compositions to implement methods of the invention. In some aspects, kits can be used to evaluate one or more biomarkers. In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules, antibodies, or inhibitors, or any value or range and combination derivable therein. In some aspects, there are kits for evaluating biomarker activity or level in a cell.

Kits may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

Kits for using probes, antibodies, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes antibodies that bind to such biomarkers as well as nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, antibodies, probes, and inhibitors are included in some kit aspects. In addition, a kit may include a sample that is a negative or positive control for methylation of one or more biomarkers.

It is contemplated that any method or composition described herein can be implemented with respect to any other method or composition described herein and that different aspects may be combined. The claims originally filed are contemplated to cover claims that are multiply dependent on any filed claim or combination of filed claims.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Serum Monomeric MPO in Endometriosis and Ovarian Cancer

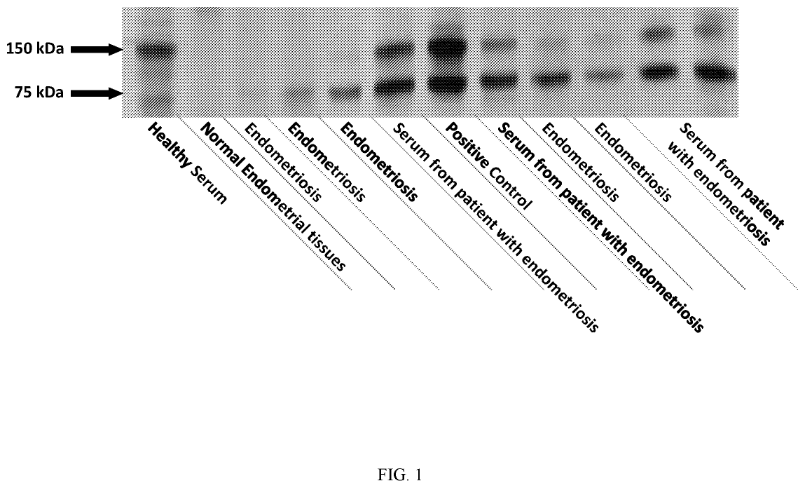

As shown in FIG. 1, the monomeric form of MPO was detected in serum obtained from patients with endometriosis but not in healthy serum which has only shown the dimeric MPO form as depicted by the 150 kDa band. As expected, no MPO is detected in normal tissues.

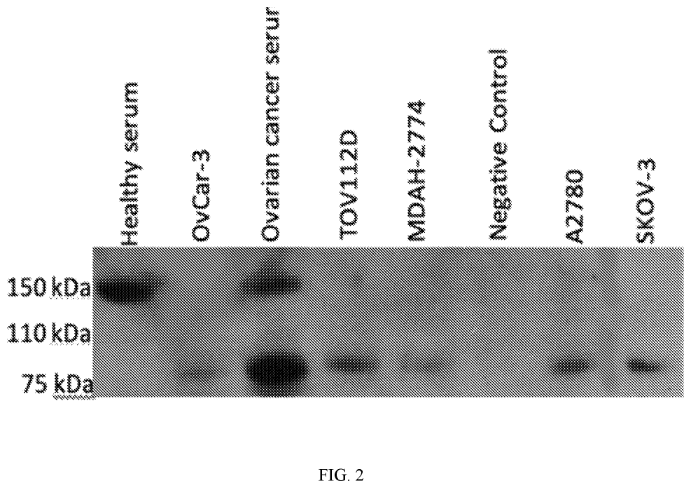

FIG. 2 shows that monomeric MPO rather than dimeric MPO is present in all ovarian cancer cells as compared to normal macrophages.

### Example 2: Monomeric Myeloperoxidase: Potential Biomarker for Early Detection of Ovarian Cancer

### A. Introduction

Ovarian cancer is a leading cause of cancer death in women, yet the underlying cause is not known [14]. Ovarian cancer originates in the epithelium of inclusion cysts, however, high-grade serous ovarian carcinoma, the most common type of ovarian cancer, is thought to originate from the fallopian tubes [3, 16]. The 5-year survival rate for non-metastatic ovarian cancer is approximately 90%, it drops to about 60% for regional disease and about 20% for metastatic disease [9]. The lack of an early-stage screening methods with high specificity, specificity, or both, significantly contributes to the observed high mortality. Moreover, early-stage ovarian cancer presents with nonspecific symptoms, thus diagnosis is frequently made after the malignancy has spread beyond the ovaries [18].

Ovarian cancer cells are characterized by an enhanced oxidative stress environment which is further increased in chemoresistance ovarian cancer [8]. The inventor was the first to report that myeloperoxidase (MPO), a key oxidant enzyme, is expressed by ovarian cancer cells and tissues [23]. This finding was surprising as MPO, an abundant hemoprotein, known to be present solely in neutrophils and monocytes and play an essential role in immune surveillance and host defense mechanisms [19]. The inventor has reported a cross-talk between MPO and inducible nitric oxide synthase (iNOS), a key pro-oxidant enzyme, as a key mechanism of decreased apoptosis in ovarian cancer cells [23]. Additional findings from the inventor's laboratory highlighted the potential benefits of the combination of serum MPO and free iron as biomarkers for early detection and prognosis of ovarian cancer [9].

Mature MPO is a ˜150 kDa symmetric, glycosylated homodimer. MPO monomer, encoded by a single gene on chromosome 17, is made of 59 kDa heavy (α) and 13.5 kDa light (β) subunits, covalently linked by disulfide bonds [11]. The formation of mature glycosylated heme-containing dimeric MPO from monomeric pro-MPO is a complex process involving a number of proteolytic reactions and post-translational modifications [31]. Additionally, MPO expression levels depend upon allelic polymorphisms in the promoter region. Particularly, a substitution of G to A at position −463 (G-463A) leads to a 25-fold decrease in MPO transcription [22]. In fact, this MPO single nucleotide polymorphism (SNP) has been reported with increased risk of breast and ovarian cancers [4,5,32]. The primary function of mature MPO, an oxidant-producing enzyme, is to catalyze the formation of hypochlorous acid (HOCl), a powerful antimicrobial agent. Mature MPO is then decomposed by HOCl into monomers (MPO-Cl). Monomeric MPO was reported to be detected in the plasma of patients with acute inflammation [12]. The impact of MPO dimerization on structural and functional properties of MPO is not fully understood.

In this study, the inventor demonstrated that ovarian cancer cells express only the monomer form of MPO which discriminated between early and late stage of the disease. This novel finding emphasizes MPO as a biomarker that is critically needed for early detection of this disease.

### B. Methods

### 1. Patient Population:

Sera (n=15) were collected from patients presenting to the gynecologic oncology division of Karmanos Cancer Institute with suspected EOC were invited to participate in a prior study. They underwent informed consent (Wayne State University Human Subject Committee protocol number 027201MP2E) and agreed to provide a blood sample prior to treatment (chemotherapy or surgery). Cases include early through late-stage diagnoses as well as a variety of histologies. Stage I is designated as early stage, as compared to remaining stages II through IV (II-IV).

Benign controls: Sera (n=14) from patients with benign gynecologic conditions was acquired through the Cooperative Human Tissue Network (CHTN). These include women diagnosed with ovarian cysts, peritonitis (inflammation), or uterine fibroids.

Healthy control sera (n=8) were procured from women recruited through a local community organization. These women presented as healthy, with no history of cancer. Basic information such as age, race, and evidence of benign gynecologic conditions, if any, was obtained at the time of informed consent. The age and racial makeup of this group overlaps with patients with ovarian cancer and benign conditions but is not explicitly matched.

### 2. Cell Lines and Cell Culture:

The human cell lines: MDAH-2774, OvCar-3, OV-21, OV-90, TOV112D (a kind gift from Gen Sheng Wu at Wayne State University, Detroit, Michigan), SKOV-3, A2780, CRL-1671, BXPC-3, COLO-3, DLCL-2, and HTB-4 were obtained from American Type Culture Collection (ATCC, Manassas, VA). Cell lines were cultured in 75 cm² cell culture flasks (Corning Incorporated, Corning, NY) with their respective media complying with the manufacturer protocol. Media is supplemented with 100 U/mL penicillin and 100 μg/mL streptomycin including 10% heat-inactivated FBS at 37° C. in 5% CO₂. Culture medium was replaced every two days. For each experiment, cells were plated in 60 mm×15 mm cell culture dishes at a cell density of approximately 2×10⁶ cells per dish and cultured for another 24 hours before collection of RNA and protein. All experiments were performed in triplicate.

### 3. Myeloperoxidase Enzyme Immunometric Assay:

Monomeric MPO was purified from all samples by gel filtration on Sephacryl S-200 HR to remove the traces of dimeric MPO before running ELISA. Myeloperoxidase Enzyme Immunometric Assay Kit (Assay Designs Catalog No. 900-115). The inventor has utilized Assay Designs' human Myeloperoxidase Enzyme Immunometric Assay (EIA) kit, a well-established assay in the inventor's laboratory, according to the manufacturer's protocol. Briefly, the kit uses a monoclonal antibody to MPO immobilized on a microtiter plate to bind the MPO in the standards or sample. A native MPO Standard is provided in the kit. Rabbit polyclonal antibody to MPO is added and binds to the MPO captured on the plate followed by the addition of goat anti-rabbit IgG conjugated to horseradish peroxidase, which binds to the polyclonal MPO antibody. The enzyme reaction is stopped, and the color generated is read at 450 nm. The measured optical density is directly proportional to the concentration of MPO in either standards or samples. The sensitivity of the assay, defined as the concentration of human MPO was determined to be 0.019 ng/mL.

### 4. Real-Time Reverse Transcriptase Polymerase Chain Reaction (RT-PCR) for MPO:

Total RNA was isolated from ovarian cancer cell lines utilizing a monophasic solution of phenol and GITC/Trizol following the previously described method [24-27]. Quantification of the RNA samples was performed using a Nanodrop spectrophotometer (Thermo Fisher Scientific, Waltham, Massachusetts).

Preparation of complimentary DNA (cDNA) was performed as follows: A 20 μl reaction volume including 1 μg total RNA, 1 μl oligo (dT, 500 μg/ml; Invitrogen, Waltham, MA), and 1 μl 10 mM dNTP mix (Invitrogen, Waltham, MA) were heated to 65° C. for 5 minutes, and then quickly chilled on ice. A master mixture containing 4 μl 5× First Strand Buffer, 2 μl 0.1M DTT, and 1 μl RNaseOut Recombinant Ribonuclease Inhibitor (40 units/μl; Invitrogen, Waltham, MA) was added and incubated at 42ºC for 2 minutes. 1 μl (200 units) of SuperScript II (Invitrogen, Waltham, MA) was added to each reaction and incubated for 50 minutes at 42ºC. Finally, the enzyme was inactivated by heating at 70° C. for 15 minutes.

Quantitative RT-PCR was performed using a Express SYBR Green RT-PCR kit (Life Technologies, Grand Island, New York) and Cepheid 1.2f Detection System (Cepheid, Sunnyvale, CA). A 25 μl total reaction volume included 12.5 μl of 2× QuantiTect SYBR Green RT-PCR master mix, 3 μl of cDNA template, and 0.2 μM each of target specific primers designed to amplify a part of each gene. Optimal oligonucleotide primer for real-time RT-PCR amplification of reverse-transcribed cDNA was selected with the aid of software program Oligo 4.0 (National Bioscience Inc., Plymouth, MN). The sequence utilized for MPO (NM_000250) is as follows: sense (5′-3′) CACTTGTATCCTCTGGTTCTTCAT (SEQ ID NO:1) and antisense (3′-5′) TCTATATGCTTCTCACGCCTAGTA (SEQ ID NO:2) with a 79 bp standard. For β-actin (NM_001101): sense (5′-3′) ATGACTTAGTTGCGTTACAC (SEQ ID NO:3) and antisense (3′-5′) AATAAAGCCATGCCAATCTC (SEQ ID NO:4) with a 79 bp standard. PCR reaction conditions were programmed as follows: An initial cycle was performed at 95ºC for 60 seconds, followed by 35 cycles of denaturation at 95° C. for 15 seconds, annealing at 60ºC for 63 seconds (for MPO) and 58° C. for 10 seconds (β-actin). This was followed by a final cycle at 72ºC for 30 seconds to allow completion of product synthesis.

To quantify each target transcript, a standard curve was constructed with serial dilutions of standard plasmid (Invitrogen, Waltham, MA). After PCR, a melting curve analysis was performed to demonstrate the specificity of the PCR product as a single peak. The control, containing all the reaction components except for the template, was included in all experiments. The amount of mRNA was then normalized to the abundance of a housekeeping gene, β-actin.

To evaluate the validity of using β-actin as an internal standard and changes in the amounts of β-actin, mRNA was tested as an external standard. Subsequently, the normalized values of the mRNA were divided by those in controls. Student's unpaired t test was used for group comparisons.

### 5. Receiver Operating Characteristic (ROC):

The inventor estimated the diagnostic ability using receiver operating characteristic (ROC) curves and confidence bands [10, 21]. ROC curves allow assessment of sensitivity and specificity of a continuous biomarker over the full range of potential cutoffs.

### 6. Western Blot:

Total proteins were isolated from various human ovarian cancer cells, (SKOV-3, A2780, OvCar-3, MDAH-2774, and TOV112D), sera from ovarian cancer patients, and healthy volunteers. Total proteins (50 μg) were subjected to non-reducing western blot and MPO was detected using a monoclonal MPO antibody (Santa Cruz Biotechnology, Dallas, TX) as previously described [7, 15, 20, 28].

### 7. Statistical Analysis:

Data was analyzed using SPSS 19.0 for windows (SPSS for Windows, Chicago, IL). Data was analyzed using oneway ANOVA (analysis of variance) with Student Neuman-Kuels post-hoc comparisons. Significance values of p<0.05 were considered statistically significant for all analyses.

### C. Results

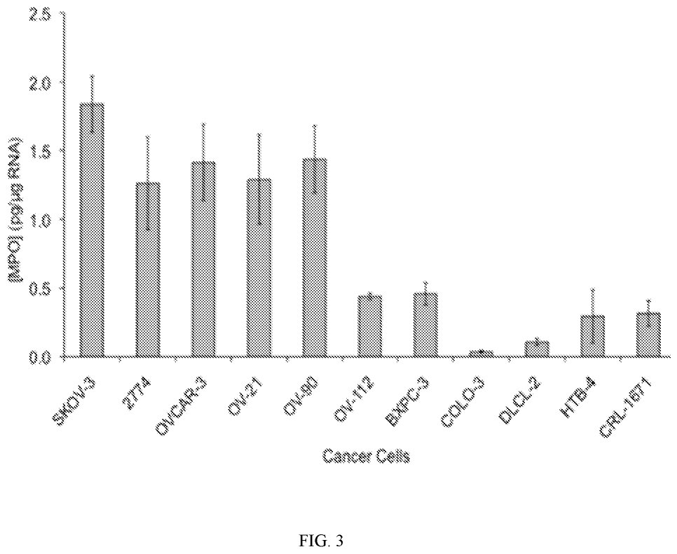

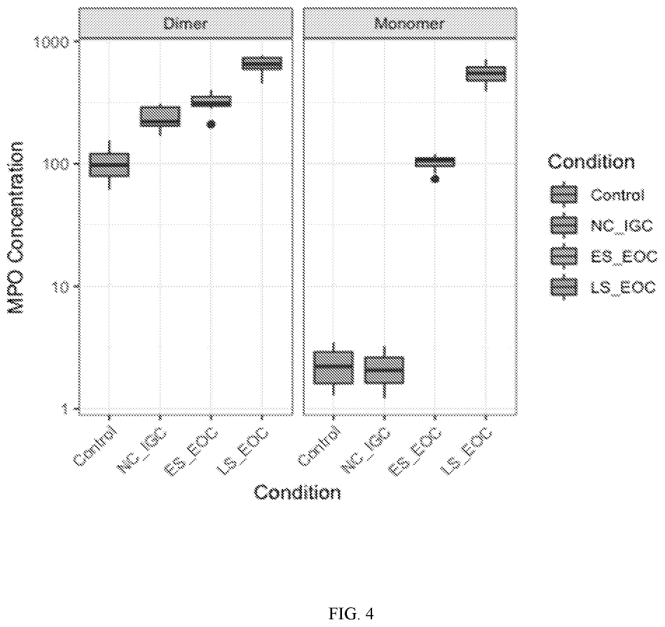

In this study the inventor has demonstrated the presence of only the monomer form of MPO in cells from ovarian cancer (FIG. 2). Both the monomer and dimer forms of MPO are present in serum from ovarian cancer patients, with markedly higher monomer MPO levels (FIG. 2). However, only the dimer MPO form was detected in the serum of healthy controls (FIG. 2). Additionally, the expression of MPO is unique to ovarian cancer cells, as it was not detected in several other types of cancer cell lines (FIG. 3). More importantly, the inventor has demonstrated the ability of serum monomer MPO to discriminate between early-stage ovarian cancer, healthy controls, and inflammatory benign gynecologic disorders, such as peritonitis and fibroids (FIG. 4, 5, p<0.05). Furthermore, serum monomer MPO discriminated early-stage from late-stage ovarian cancer (FIG. 4, 5, p<0.05).

### D. Discussion

The inventor's laboratory has been studying the role of oxidative stress and inflammation in the pathogenesis of ovarian cancer for over two decades. Indeed, he was the first to demonstrate that myeloperoxidase is present in all ovarian cancer cell lines and tissues, with minimal or no presence detected in normal ovarian tissues [23]. Additionally, crosstalk between MPO and inducible nitric oxide synthase (iNOS), a key pro-oxidant enzyme, has proven to be a vital mechanism of decreased apoptosis in EOC cells [23]. In this mechanism, MPO utilizes nitric oxide (NO), produced by iNOS, as a one-electron substrate that generates nitrosonium cation (NO⁺), a labile nitro sating species, that increases S-nitrosylation of caspase-3, inhibiting its activity and thus decreasing apoptosis, a hallmark of ovarian cancer [2].

The inventor has previously demonstrated the potential benefits of the combination of serum MPO and free iron as biomarkers for early detection and prognosis of ovarian cancer in small number of patients [9]. To date, MPO is not validated as a biomarker for early detection and prognosis of ovarian cancer. In this study the inventor has demonstrated that MPO exists in its monomeric form in all ovarian cancer cells tested (FIG. 2). The reason for this phenomenon remains unknown and is currently under investigation. A possible explanation is attributed to the enhanced intrinsic oxidative environment that characterizes ovarian cancer cells [13,29]. The fact that the monomeric form of MPO is detected in the blood of ovarian cancer patients but not in healthy individuals emphasizes its potential use as a biomarker for this disease. Indeed, these results have clearly shown monomeric MPO to significantly discriminate between healthy conditions and benign inflammatory conditions, early-stage, and late-stage ovarian cancer (FIG. 4, 5).

Due to the low prevalence of ovarian cancer in U.S. women, an ovarian cancer diagnostic or screening test must have a minimum specificity of 99.6% before it can be used routinely in the general population of postmenopausal women [6,17]. Such a test may offset potential morbidity and mortality, which can be associated with complications of surgery for patients who have false-positive ovarian cancer screening tests [6, 17]. An ovarian cancer screening test should also have high sensitivity and a suitable positive predictive value (PPV) [17, 18]. Routine screening for ovarian cancer in the general population is not recommended because traditional screening methods are not sensitive or specific enough [1]. Therefore, the development of sensitive and specific methods for early detection has been a priority as a means for improving the diagnosis and treatment of this disease.

To conclude, the concept of using monomeric serum MPO levels as a way to infer the source of the MPO, and thus discriminate between ovarian cancer-associated and inflammation-associated MPO is novel. If the findings that were determined in a small set of ovarian tumors is confirmed in a larger set, a valuable biomarker will be identified. The result from this study emphasizes MPO as a potential biomarker that is critically needed for early detection of this disease.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and the references cited throughout the specification, to the extent that they provide exemplary procedural or other details supplementary to those set forth herein, are specifically incorporated herein by reference.
[1] Screening for Ovarian Cancer: Recommendation Statement. Am Fam Physician 2018; 97(12): Online.
[2] Abu-Soud H M, Hazen S L. Nitric oxide modulates the catalytic activity of myeloperoxidase. J Biol Chem 2000; 275(8): 5425-30.
[3] Auersperg N, Wong A S, Choi K C, Kang S K, Leung P C. Ovarian surface epithelium: biology, endocrinology, and pathology. Endocr Rev 2001; 22(2): 255-88.
[4] Belotte J, Fletcher N M, Saed M G, Abusamaan M S, Dyson G, Diamond M P et al. A Single Nucleotide Polymorphism in Catalase Is Strongly Associated with Ovarian Cancer Survival. PLOS One 2015; 10(8): e0135739.
[5] Castillo-Tong D C, Pils D, Heinze G, Braicu I, Sehouli J, Reinthaller A et al. Association of myeloperoxidase with ovarian cancer. Tumour Biol 2014; 35(1): 141-8.
[6] Clarke-Pearson D L. Clinical practice. Screening for ovarian cancer. N Engl J Med 2009; 361(2): 170-7.
[7] Fletcher N M, Abuanzeh S, Saed M G, Diamond M P, Abu-Soud H M, Saed G M. Nicotinamide Adenine Dinucleotide Phosphate Oxidase Expression Is Differentially Regulated to Favor a Pro-oxidant State That Contributes to Postoperative Adhesion Development. Reprod Sci 2014; 21(8): 1050-1059.
[8] Fletcher N M, Belotte J, Saed M G, Memaj I, Diamond M P, Morris R T et al. Specific point mutations in key redox enzymes are associated with chemoresistance in epithelial ovarian cancer. Free Radic Biol Med 2017; 102: 122-132.
[9] Fletcher N M, Jiang Z, Ali-Fehmi R, Levin N K, Belotte J, Tainsky M A et al. Myeloperoxidase and free iron levels: potential biomarkers for early detection and prognosis of ovarian cancer. Cancer Biomark 2011; 10(6): 267-75.
[10] Floris R, Wever R. Reaction of myeloperoxidase with its product HOCl. Eur J Biochem 1992; 207(2): 697-702.
[11] Furtmuller P G, Jantschko W, Regelsberger G, Jakopitsch C, Moguilevsky N, Obinger C. A transient kinetic study on the reactivity of recombinant unprocessed monomeric myeloperoxidase. FEBS Lett 2001; 503(2-3): 147-50.
[12] Gorudko I V, Grigorieva D V, Sokolov A V, Shamova E V, Kostevich V A, Kudryavtsev I V et al. Neutrophil activation in response to monomeric myeloperoxidase. Biochem Cell Biol 2018; 96(5): 592-601.
[13] Hileman E O, Liu J, Albitar M, Keating M J, Huang P. Intrinsic oxidative stress in cancer cells: a biochemical basis for therapeutic selectivity. Cancer Chemother Pharmacol 2004; 53(3): 209-19
[14] Jemal A, Siegel R, Ward E, Murray T, Xu J, Smigal C et al. Cancer statistics, 2006. CA Cancer J Clin 2006; 56(2): 106-30.
[15] Jiang Z L, Fletcher N M, Diamond M P, Abu-Soud H M, Saed G M. Hypoxia regulates iNOS expression in human normal peritoneal and adhesion fibroblasts through nuclear factor kappa B activation mechanism. Fertil Steril 2009; 91(2): 616-21.
[16] Kim J, Park E Y, Kim O, Schilder J M, Coffey D M, Cho C H et al. Cell Origins of High-Grade Serous Ovarian Cancer. Cancers (Basel) 2018; 10(11).
[17] Leahy Y. Are serum protein biomarkers effective in detecting ovarian cancer in its early stages? Clin J Oncol Nurs 2009; 13(4): 443-5.
[18] MacDonald N D, Rosenthal A N, Jacobs I J. Screening for ovarian cancer. Ann Acad Med Singap 1998; 27(5): 676-82.
[19] Ndrepepa G. Myeloperoxidase—A bridge linking inflammation and oxidative stress with cardiovascular disease. Clin Chim Acta 2019; 493: 36-51.
[20] Nusrat O, Belotte J, Fletcher N M, Memaj I, Saed M G, Diamond M P et al. The Role of Angiogenesis in the Persistence of Chemoresistance in Epithelial Ovarian Cancer. Reprod Sci 2016; 23(11): 1484-1492.
[21] Pepe M S, Etzioni R, Feng Z, Potter J D, Thompson M L, Thornquist M et al. Phases of biomarker development for early detection of cancer. J Natl Cancer Inst 2001; 93(14): 1054-61.
[22] Piedrafita F J, Molander R B, Vansant G, Orlova E A, Pfahl M, Reynolds W F. An Alu element in the myeloperoxidase promoter contains a composite SP1-thyroid hormone-retinoic acid response element. J Biol Chem 1996; 271(24): 14412-20.
[23] Saed G M, Ali-Fehmi R, Jiang Z L, Fletcher N M, Diamond M P, Abu-Soud H M et al. Myeloperoxidase serves as a redox switch that regulates apoptosis in epithelial ovarian cancer. Gynecol Oncol 2010; 116(2): 276-81.
[24] Saed G M, Diamond M P. Apoptosis and proliferation of human peritoneal fibroblasts in response to hypoxia. Fertil Steril 2002; 78(1): 137-43.
[25] Saed G M, Diamond M P. Hypoxia-induced irreversible up-regulation of type I collagen and transforming growth factor-beta1 in human peritoneal fibroblasts. Fertil Steril 2002; 78(1): 144-7.
[26] Saed G M, Diamond M P. Modulation of the expression of tissue plasminogen activator and its inhibitor by hypoxia in human peritoneal and adhesion fibroblasts. Fertil Steril 2003; 79(1): 164-8
[27] Saed G M, Diamond M P. Molecular characterization of postoperative adhesions: the adhesion phenotype. J Am Assoc Gynecol Laparosc 2004; 11(3): 307-14.
[28] Saed G M, Fletcher N M, Diamond M P, Morris R T, Gomez-Lopez N, Memaj I. Novel expression of CD11b in epithelial ovarian cancer: Potential therapeutic target. Gynecol Oncol 2018; 148(3): 567-575.
[29] Saed G M, Fletcher N M, Jiang Z L, Abu-Soud H M, Diamond M P. Dichloroacetate induces apoptosis of epithelial ovarian cancer cells through a mechanism involving modulation of oxidative stress. Reprod Sci 2011; 18(12): 1253-61.
[30] Saed G M, Jiang Z, Diamond M P, Abu-Soud H M. The role of myeloperoxidase in the pathogenesis of postoperative adhesions. Wound Repair Regen 2009; 17(4): 531-9.
[31] Van Antwerpen P, Slomianny M C, Boudjeltia K Z, Delporte C, Faid V, Calay D et al. Glycosylation pattern of mature dimeric leukocyte and recombinant monomeric myeloperoxidase: glycosylation is required for optimal enzymatic activity. J Biol Chem 2010; 285(21): 16351-9.
[32] Wheatley-Price P, Asomaning K, Reid A, Zhai R, Su L, Zhou W et al. Myeloperoxidase and superoxide dismutase polymorphisms are associated with an increased risk of developing pancreatic adenocarcinoma. Cancer 2008; 112(5): 1037-42.
Screening for Ovarian Cancer: Recommendation Statement. Am Fam Physician 2018; 97(12): Online.

1. A method for evaluating a subject comprising detecting monomeric myeloperoxidase (MPO) in a biological sample from the subject.

2. The method of claim 1, wherein the biological sample comprises serum, plasma, or tissue sample.

3. The method of claim 2, wherein the biological sample comprises a serum or plasma sample.

4. The method of any one of claims 1-3, wherein the method further comprises detecting iron levels in the biological sample from the subject.

5. The method of claim 4, wherein detecting iron levels in the biological sample from the subject comprises using one or more of a serum iron test, a free iron test, a total iron-binding capacity (TIBC) test, or a ferritin test.

6. The method of any one of claims 1-5, wherein the method comprises performing size fractionation of the biological sample and wherein size fractionation provides a fraction of the biological sample that comprises molecules that are 75 kDa and excludes molecules that are 150 kDa or larger.

7. The method of claim 6, wherein the method comprises detecting monomeric MPO in the fraction comprising molecules that are 75 kDa.

8. The method of any one of claims 1-7, wherein detecting monomeric MPO comprises using one or more antibodies that specifically binds monomeric MPO.

9. The method of any one of claims 1-8, wherein detecting monomeric MPO comprises an ELISA assay.

10. The method of any one of claims 1-9, wherein detecting monomeric MPO comprises contacting the biological sample or the fraction comprising molecules that are 75 kDa with an anti-MPO antibody or MPO binding molecule under conditions that allow for the binding of MPO to the anti-MPO antibody.

11. The method of claim 10, wherein the anti-MPO antibody or binding molecule is linked to a solid support.

12. The method of claim 11, wherein the method further comprises washing the solid support to remove unbound molecules.

13. The method of any one of claims 1-12, wherein the method comprises or further comprises contacting the biological sample or the fraction with a capture antibody or antigen-binding molecule.

14. The method of claim 13, wherein the capture antibody or antigen-binding molecule comprise a second anti-MPO antibody or MPO antigen-binding fragment.

15. The method of claim 13 or 14, wherein the capture antibody is linked to a detectable label.

16. The method of claim 15, wherein the method further comprises quantitatively or qualitatively evaluating the detectable label.

17. The method of any one of claims 1-16 wherein the subject is one that has one or more symptoms of endometriosis and/or ovarian cancer.

18. The method of any one of claims 1-17, wherein the subject has been diagnosed with an endometrial or ovarian hyperproliferative disorder.

19. The method of any one of claims 1-18, wherein the subject has been treated for an endometrial or ovarian hyperproliferative disorder, will be treated for an endometrial or ovarian hyperproliferative disorder, or is currently undergoing treatment for an endometrial or ovarian hyperproliferative disorder.

20. The method of any one of claims 1-19, wherein the subject is female.

21. The method of any one of claims 1-20, wherein the subject is on hormone therapy.

22. The method of claim 21, wherein the hormone therapy comprises contraception or hormone replacement therapy.

23. The method of any one of claims 20-22, wherein the female is an adolescent, perimenopausal, or menopausal female.

24. The method of any one of claims 1-23, wherein the method further comprises quantitating the level of monomeric MPO in the biological sample.

25. The method of claim 24, wherein the level of monomeric MPO is normalized.

26. The method of claim 24 or 25, wherein the level of monomeric MPO is compared to a control.

27. The method of any one of claims 24-26, wherein the level of monomeric MPO is determined to be greater than the control.

28. The method of any one of claims 24-26, wherein the level of monomeric MPO is determined to be less than the control.

29. The method of any one of claims 1-28, wherein the control comprises the level of monomeric MPO that is representative of the level of monomeric MPO in a biological sample from a subject with endometriosis.

30. The method of any one of claims 1-28, wherein the control comprises the level of monomeric MPO that is representative of the level of monomeric MPO in a biological sample from a subject with ovarian cancer.

31. The method of any one of claims 1-28, wherein the control comprises the level of monomeric MPO that is representative of the level of monomeric MPO in a biological sample from a subject without an endometrial or ovarian hyperproliferative disorders.

32. The method of any one of claims 1-31, wherein the method further comprises diagnosing the subject.

33. The method of claim 32, wherein the subject is diagnosed with ovarian cancer based on the determined level of MPO.

34. The method of claim 33, wherein the subject is diagnosed with stage I, II, III, or IV ovarian cancer based on the determined level of MPO.

35. The method of claim 33 or 34, wherein the method further comprises treating the subject for ovarian cancer.

36. The method of claim 32, wherein the subject is diagnosed with endometriosis based on the determined level of MPO.

37. The method of claim 36, wherein the method further comprises treating the subject for endometriosis.

38. A method for making a complex comprising contacting a biological sample with an antibody that binds to monomeric MPO.

39. The method of claim 38, wherein the biological sample comprises serum, plasma, or tissue sample.

40. The method of claim 39, wherein the biological sample comprises a serum or plasma sample.

41. The method of any one of claims 38-40, wherein the biological sample comprises a size-fractionated biological sample wherein the size-fractionated biological sample comprises molecules that are 75 kDa and excludes molecules that are 150 kDa or larger.

42. The method of any one of claims 38-41, wherein contacting a biological sample with an antibody that binds to monomeric MPO comprises performing an ELISA assay.

43. The method of any one of claims 38-42, wherein the method comprises contacting the biological sample or the fraction comprising molecules that are 75 kDa with an anti-MPO antibody or MPO binding molecule under conditions that allow for the binding of MPO to the anti-MPO antibody.

44. The method of claim 43, wherein the anti-MPO antibody or binding molecule is linked to a solid support.

45. The method of claim 44, wherein the method further comprises washing the solid support to remove unbound molecules.

46. The method of any one of claims 38-45, wherein the method comprises or further comprises contacting the biological sample or the fraction with a capture antibody or antigen-binding molecule.

47. The method of claim 46, wherein the capture antibody or antigen-binding molecule comprises a second anti-MPO antibody or MPO antigen-binding fragment.

48. The method of claim 46 or 47, wherein the capture antibody is linked to a detectable label.

49. The method of claim 48, wherein the method further comprises quantitatively or qualitatively evaluating the detectable label.

50. The method of any one of claims 38-49 wherein the biological sample is from a subject that has one or more symptoms of endometriosis and/or ovarian cancer.

51. The method of any one of claims 38-50, wherein the biological sample is from a subject that has been diagnosed with an endometrial or ovarian hyperproliferative disorder.

52. The method of any one of claims 38-51, wherein the biological sample is from a subject that has been treated for an endometrial or ovarian hyperproliferative disorder, will be treated for an endometrial or ovarian hyperproliferative disorder, or is currently undergoing treatment for an endometrial or ovarian hyperproliferative disorder.

53. The method of any one of claims 38-52, wherein the biological sample is from a female subject.

54. The method of any one of claims 38-53, wherein the biological sample is from a subject on hormone therapy.

55. The method of claim 54, wherein the hormone therapy comprises contraception or hormone replacement therapy.

56. The method of any one of claims 53-55, wherein the female is an adolescent, perimenopausal, or menopausal female.

57. The method of any one of claims 38-56, wherein the method further comprises quantitating the level of monomeric MPO in the biological sample.

58. The method of claim 57, wherein the level of monomeric MPO is normalized.

59. The method of claim 57 or 58, wherein the level of monomeric MPO is compared to a control.

60. The method of any one of claims 57-59, wherein the level of monomeric MPO is determined to be greater than the control.

61. The method of any one of claims 57-59, wherein the level of monomeric MPO is determined to be less than the control.

62. The method of claim 60 or 61, wherein the control comprises the level of monomeric MPO that is representative of the level of monomeric MPO in a biological sample from a subject with endometriosis.

63. The method of claim 60 or 61, wherein the control comprises the level of monomeric MPO that is representative of the level of monomeric MPO in a biological sample from a subject with ovarian cancer.

64. The method of claim 60 or 61, wherein the control comprises the level of monomeric MPO that is representative of the level of monomeric MPO in a biological sample from a subject without an endometrial or ovarian hyperproliferative disorders.

65. A method for treating a subject with an endometrial or ovarian hyperproliferative disorder, the method comprising administering an treatment for the endometrial or ovarian hyperproliferative disorder to a subject that has had the level of monomeric MPO evaluated in a biological sample from the subject.

66. The method of claim 65, wherein the biological sample comprises serum, plasma, or tissue sample.

67. The method of claim 65 or 66, wherein the subject is female.

68. The method of any one of claims 65-67, wherein the subject is on hormone therapy.

69. The method of claim 68, wherein the hormone therapy comprises contraception or hormone replacement therapy.

70. The method of any one of claims 67-69, wherein the female is an adolescent, perimenopausal, or menopausal female.

71. The method of any one of claims 65-70, wherein the level of monomeric MOP in the biological sample from the subject has been quantitated.

72. The method of claim 71, wherein the level of monomeric MPO is normalized.

73. The method of any one of claims 65-72, wherein the subject has or has been determined to have a level of monomeric MPO in the biological sample that is greater than the level of a monomeric MPO in a control sample.

74. The method of any one of claims 65-72, wherein the subject has or has been determined to have a level of monomeric MPO in the biological sample that is less than the level of a monomeric MPO in a control sample.

75. The method of any one of claims 65-72, wherein the subject has or has been determined to have a level of monomeric MPO in the biological sample that is not significantly different than the level of a monomeric MPO in a control sample.

76. The method of any one of claims 65-75, wherein the control comprises the level of monomeric MPO that is representative of the level of monomeric MPO in a subject with endometriosis.

77. The method of any one of claims 65-75, wherein the control comprises the level of monomeric MPO that is representative of the level of monomeric MPO in a subject with ovarian cancer.

78. The method of any one of claims 65-75, wherein the control comprises the level of monomeric MPO that is representative of the level of monomeric MPO in a subject without an endometrial or ovarian hyperproliferative disorders.

79. The method of any one of claims 65-78, wherein the subject has been evaluated for iron levels.

80. The method of claim 79, wherein the subject has been evaluated for iron levels by having one or more of a serum iron test, a free iron test, a total iron-binding capacity (TIBC) test, or a ferritin test in a biological sample from the subject.

81. The method of claim 79 or 80, wherein the subject has been determined to have abnormal iron levels.

82. The method of claim 81, wherein the subject has been determined to have a lower than normal TIBC or higher than normal ferritin.

83. The method of claim 81, wherein the amount of free iron was determined to be higher than normal in the biological sample from the subject.

84. The method of any one of claims 65-83, wherein the hyperproliferative disorder comprises endometriosis.

85. The method of claim 84, wherein the treatment comprises hormone therapy, or surgery.

86. The method of any one of claims 65-82, wherein the hyperproliferative disorder comprises ovarian cancer.

87. The method of claim 86, wherein the cancer comprises with stage I, II, III, or IV ovarian cancer.

88. The method of claim 86 or 87, wherein the treatment comprises surgery, radiation, chemotherapy, hormone therapy, or targeted therapy.

89. A method of diagnosing or prognosing a subject with an endometrial or ovarian hyperproliferative disorder comprising
a) evaluating monomeric MPO in a biological sample from the subject;
b) comparing the measured level to control level or control samples; and
c) diagnosing or prognosing the subject with an endometrial or ovarian hyperproliferative disorder based on the measured level of monomeric MPO.

90. The method of claim 89, wherein the biological sample comprises serum, plasma, or tissue sample.

91. The method of claim 90, wherein the biological sample comprises a serum or plasma sample.

92. The method of any one of claims 89-91, wherein the method further comprises evaluating iron levels in a biological sample from the subject.

93. The method of claim 92, wherein detecting iron levels in the biological sample from the subject comprises using one or more of a serum iron test, a free iron test, a total iron-binding capacity (TIBC) test, or a ferritin test.

94. The method of any one of claims 89-91, wherein the method comprises size fractionation of the biological sample and wherein size fractionation provides a fraction or the biological sample that comprises molecules that are 75 kDa and excludes molecules that are 150 kDa or larger.

95. The method of claim 94, wherein the method comprises evaluating monomeric MPO in the fraction comprising molecules that are 75 kDa.

96. The method of any one of claims 89-95, wherein evaluating monomeric MPO comprises using one or more antibodies that specifically bind monomeric MPO.

97. The method of any one of claims 89-96, wherein evaluating monomeric MPO comprises an ELISA assay.

98. The method of any one of claims 89-97, wherein evaluating monomeric MPO comprises contacting the biological sample or the fraction comprising molecules that are 75 kDa with an anti-MPO antibody or MPO binding molecule under conditions that allow for the binding of MPO to the anti-MPO antibody.

99. The method of claim 98, wherein the anti-MPO antibody or binding molecule is linked to a solid support.

100. The method of claim 99, wherein the method further comprises washing the solid support to remove unbound molecules.

101. The method of any one of claims 89-100, wherein the method comprises or further comprises contacting the biological sample or the fraction with a capture antibody or antigen-binding molecule.

102. The method of any one of claims 89-101, wherein evaluating monomeric MPO comprises quantitating the level of monomeric MPO in the biological sample.

103. The method of claim 102, wherein the subject is diagnosed as not having an endometrial or ovarian hyperproliferative disorder when monomeric MPO: i) is not detected in the biological sample from the subject; ii) is not significantly different than a control, wherein the control comprises the level of monomeric MPO that is representative of the level of monomeric MPO in a biological sample from a subject without an endometrial or ovarian hyperproliferative disorders; or iii) is less than a control, wherein the control comprises the level of monomeric MPO that is representative of the level of monomeric MPO in a biological sample from a subject with an endometrial or ovarian hyperproliferative disorder.

104. The method of claim 102, wherein the subject is diagnosed as not having an endometrial or ovarian hyperproliferative disorder when
(A) monomeric MPO:
i) is not detected in the biological sample from the subject;
ii) is not significantly different than a control, wherein the control comprises the level of monomeric MPO that is representative of the level of monomeric MPO in a biological sample from a subject without an endometrial or ovarian hyperproliferative disorders; or
iii) is less than a control, wherein the control comprises the level of monomeric MPO that is representative of the level of monomeric MPO in a biological sample from a subject with an endometrial or ovarian hyperproliferative disorder; and
(B) when iron levels are normal.

105. The method of claim 102, wherein the subject is diagnosed as having endometriosis when monomeric MPO: i) is greater than a control, wherein the control comprises the level of monomeric MPO that is representative of the level of monomeric MPO in a biological sample from a subject without an endometrial or ovarian hyperproliferative disorders; ii) is not significantly different than the control; wherein the control comprises the level of monomeric MPO that is representative of the level of monomeric MPO in a biological sample from a subject with endometriosis; or iii) is less than a control, wherein the control comprises the level of monomeric MPO that is representative of the level of monomeric MPO in a biological sample from a subject with ovarian cancer.

106. The method of claim 102, wherein the subject is diagnosed as having endometriosis when
(A) monomeric MPO:
i) is greater than a control, wherein the control comprises the level of monomeric MPO that is representative of the level of monomeric MPO in a biological sample from a subject without an endometrial or ovarian hyperproliferative disorders;
ii) is not significantly different than the control; wherein the control comprises the level of monomeric MPO that is representative of the level of monomeric MPO in a biological sample from a subject with endometriosis; or
iii) is less than a control, wherein the control comprises the level of monomeric MPO that is representative of the level of monomeric MPO in a biological sample from a subject with ovarian cancer; and
(B) when iron levels are abnormal.

107. The method of claim 106, wherein the TIBC is lower than normal and/or ferritin is higher than normal in the biological sample from the subject.

108. The method of claim 106 or 107, wherein the amount of free iron is higher than normal in the biological sample from the subject.

109. The method of claim 102, wherein the subject is diagnosed as having ovarian cancer when monomeric MPO: i) is greater than a control, wherein the control comprises the level of monomeric MPO that is representative of the level of monomeric MPO in a biological sample from a subject without an endometrial or ovarian hyperproliferative disorders or from a subject with endometriosis; ii) is not significantly different than a control, wherein the control comprises the level of monomeric MPO that is representative of the level of monomeric MPO in a biological sample from a subject with ovarian cancer.

110. The method of claim 102, wherein the subject is diagnosed as having ovarian cancer when
(A) monomeric MPO:
i) is greater than a control, wherein the control comprises the level of monomeric MPO that is representative of the level of monomeric MPO in a biological sample from a subject without an endometrial or ovarian hyperproliferative disorders or from a subject with endometriosis; or
ii) is not significantly different than a control, wherein the control comprises the level of monomeric MPO that is representative of the level of monomeric MPO in a biological sample from a subject with ovarian cancer; and
(B) when iron levels are abnormal.

111. The method of claim 110, wherein the TIBC is lower than normal and/or ferritin is higher than normal in the biological sample from the subject.

112. The method of claim 110 or 111, wherein the amount of free iron is higher than normal in the biological sample from the subject.

113. The method of any one of claims 109-112, wherein the subject is diagnosed with stage I, II, III, or IV ovarian cancer based on the measured level of monomeric MPO.

114. The method of claim 113, wherein evaluating monomeric MPO comprises qualitatively detecting the level of monomeric MPO in the biological sample.

115. The method of claim 114, wherein the subject is diagnosed as not having an endometrial or ovarian hyperproliferative disorder when monomeric MPO in not detected in the biological sample from the subject.

116. The method of claim 113, wherein the subject is diagnosed as having an endometrial or ovarian hyperproliferative disorder when monomeric MPO is detected in the biological sample from the subject.

117. The method of any one of claims 89-116, wherein the subject is one that has one or more symptoms of endometrial or ovarian hyperproliferative disorders.

118. The method of any one of claims 89-117, wherein the subject is female.

119. The method of any one of claims 89-118, wherein the subject is on hormone therapy.

120. The method of claim 119, wherein the hormone therapy comprises contraception or hormone replacement therapy.

121. The method of any one of claims 118-120, wherein the female is an adolescent, perimenopausal, or menopausal female.

122. The method of any one of claims 102-121, wherein the level of monomeric MPO is normalized.

123. The method of any one of claim 105-107 or 116-122, wherein the method further comprises treating the subject for endometriosis.

124. The method of claim 123, wherein the treatment comprises hormone therapy, or surgery.

125. The method of any one of claims 109-122, wherein the method further comprises treating the subject for ovarian cancer.

126. The method of claim 125, wherein the treatment comprises surgery, radiation, chemotherapy, hormone therapy, or targeted therapy.

127. A method for monitoring a subject being treated for an endometrial or ovarian hyperproliferative disorder with a therapeutic agent, the method comprising
a) evaluating monomeric MPO in a biological sample from the subject;
b) comparing the measured level to control level or control samples; and
c) determining the efficacy of the therapeutic agent based on the measured level of monomeric MPO.

128. The method of claim 127, wherein the biological sample comprises serum, plasma, or tissue sample.

129. The method of claim 128, wherein the biological sample comprises a serum or plasma sample.

130. The method of any one of claims 127-129, wherein the method further comprises evaluating iron levels in a biological sample from the subject.

131. The method of claim 130, wherein detecting iron levels in the biological sample from the subject comprises using one or more of a serum iron test, a free iron test, a total iron-binding capacity (TIBC) test, or a ferritin test.

132. The method of any one of claims 127-129, wherein the method comprises size fractionation of the biological sample and wherein size fractionation provides a fraction or the biological sample that comprises molecules that are 75 kDa and excludes molecules that are 150 kDa or larger.

133. The method of claim 132, wherein the method comprises evaluating monomeric MPO in the fraction comprising molecules that are 75 kDa.

134. The method of any one of claims 127-133, wherein evaluating monomeric MPO comprises using one or more antibodies that specifically bind monomeric MPO.

135. The method of any one of claims 127-134, wherein evaluating monomeric MPO comprises an ELISA assay.

136. The method of any one of claims 127-135, wherein evaluating monomeric MPO comprises contacting the biological sample or the fraction comprising molecules that are 75 kDa with an anti-MPO antibody or MPO binding molecule under conditions that allow for the binding of MPO to the anti-MPO antibody.

137. The method of claim 136, wherein the anti-MPO antibody or binding molecule is linked to a solid support.

138. The method of claim 137, wherein the method further comprises washing the solid support to remove unbound molecules.

139. The method of any one of claims 127-138, wherein the method comprises or further comprises contacting the biological sample or the fraction with a capture antibody or antigen-binding molecule.

140. The method of any one of claims 127-139, wherein evaluating monomeric MPO comprises quantitating the level of monomeric MPO in the biological sample.

141. The method of claim 140, wherein the therapeutic agent is determined to be effective when monomeric MPO: i) is not detected in the biological sample from the subject; ii) is not significantly different than a control, wherein the control comprises the level of monomeric MPO that is representative of the level of monomeric MPO in a biological sample from a subject without an endometrial or ovarian hyperproliferative disorders; iii) is less than a control, wherein the control comprises the level of monomeric MPO that is representative of the level of monomeric MPO in a biological sample from a subject with an endometrial or ovarian hyperproliferative disorder; or iv) is decreased compared to the level of monomeric MPO before treatment of the subject with the therapeutic agent.

142. The method of claim 140, wherein the therapeutic agent is determined to be ineffective when monomeric MPO: i) detected in the biological sample from the subject; ii) is increased compared to a control, wherein the control comprises the level of monomeric MPO that is representative of the level of monomeric MPO in a biological sample from a subject without an endometrial or ovarian hyperproliferative disorders; iii) is not significantly different or more than a control, wherein the control comprises the level of monomeric MPO that is representative of the level of monomeric MPO in a biological sample from a subject with an endometrial or ovarian hyperproliferative disorder; or iv) is not significantly different or increased compared to the level of monomeric MPO before treatment of the subject with the therapeutic agent.

143. The method of any one of claims 127-142, wherein evaluating monomeric MPO comprises qualitatively detecting the level of monomeric MPO in the biological sample.

144. The method of any one of claims 127-143, wherein the subject is one that has one or more symptoms of endometrial or ovarian hyperproliferative disorders.

145. The method of any one of claims 127-144, wherein the subject has been diagnosed with an endometrial or ovarian hyperproliferative disorder.

146. The method of any one of claims 127-145, wherein the method further comprises evaluating the level of monomeric MPO in a biological sample from the subject obtained prior to treatment.

147. The method of any one of claims 127-146, wherein the method further comprises evaluating the level of monomeric MPO in a biological sample from the subject obtained after one or more treatments.

148. The method of any one of claims 127-147, wherein the subject is female.

149. The method of any one of claims 127-148, wherein the subject is on hormone therapy.

150. The method of claim 149, wherein the hormone therapy comprises contraception or hormone replacement therapy.

151. The method of any one of claims 148-150, wherein the female is an adolescent, perimenopausal, or menopausal female.

152. The method of any one of claims 140-151, wherein the level of monomeric MPO is normalized.

153. The method of any one of claims 127-152, wherein the treatment comprises hormone therapy, surgery, radiation, chemotherapy, or targeted therapy.

154. A kit comprising one or more anti-MPO antibodies or an MPO binding fragment thereof and a size exclusion column that fractionates a sample and wherein the fractionation separates polypeptides having a size of 75 kDa and polypeptide having a size of 150 kDa into separate fractions.

155. The kit of claim 154, wherein the kit further comprises one or more negative or positive control samples.

156. The kit of claim 154 or 155, wherein the kit comprises an ELISA for detecting MPO.

157. The kit of any one of claims 154-156, wherein the anti-MPO antibody or binding fragment is operatively linked to a solid support.

158. The kit of any one of claims 154-157, wherein the kit comprises at least two anti-MPO antibodies, at least two anti-MPO antibody binding fragments, or one anti-MPO antibody and one anti-MPO antibody binding fragment.

159. The kit of any one of claims 154-158, wherein the one or more anti-MPO antibodies or MPO antibody binding fragments is linked to a detectable label.


SEQUENCE LISTING (4 sequences)

SEQ ID NO: 1 - DNA, 24 bp, Artificial Sequence
  Synthetic Polynucleotide
cacttgtatcctctggttcttcat

SEQ ID NO: 2 - DNA, 24 bp, Artificial Sequence
  Synthetic Polynucleotide
tctatatgcttctcacgcctagta

SEQ ID NO: 3 - DNA, 20 bp, Artificial Sequence
  Synthetic Polynucleotide
atgacttagttgcgttacac

SEQ ID NO: 4 - DNA, 20 bp, Artificial Sequence
  Synthetic Polynucleotide
aataaagccatgccaatctc